(19) United States
(12) Patent Application Publication
(10) Pub. No.: US 2004/0219601 A1
(43) Pub. Date: Nov. 4, 2004

(54) Method and system for more effective protein three-dimensional structure prediction

(76) Inventors: Jinbo Xu, Waterloo (CA); Ming Li, Oakville (CA)

Correspondence Address: Norman P. Soloway, HAYES SOLOWAY P.C., 130 W. Cushing Street, Tucson, AZ, 85701, US

(21) Appl. No.: 10/751,230

(22) Filed: Jan. 2, 2004

(30) Foreign Application Priority Data
Jan. 2, 2003 (CA) 2,415,584

(51) Int. Cl. G01N 33/53; G06F 19/00; G01N 33/48; G01N 33/50

(52) U.S. Cl. 435/7.1; 702/19

(57) ABSTRACT

The present invention relates generally to biotechnology and information technology, and in particular, to a subfield known as bioinformatics. A specific aspect of the invention lies in the provision of a new method and system for predicting the three-dimensional structure of proteins. The main focus in this patent application is on the development of a globally optimal and practically efficient threading algorithm based on an alignment model incorporating pairwise interaction preferences explicitly, and allowing variable gaps by using an integer programming approach. Integer programming formulation can fully exploit the abovementioned special features of the pairwise interaction preferences. It allows one to use the existing powerful linear programming packages together with a branch and bound algorithm to rapidly arrive at the optimal alignment. This is the first time that integer programming and linear programming has been applied to protein threading.

The present invention relates generally to biotechnology and information technology, and in particular, to a subfield known as bioinformatics. A specific aspect of the invention lies in the provision of a new method and system for analysing the three-dimensional structure of proteins.

### BACKGROUND OF THE INVENTION

The Human Genome Project has led to the identification of over 30 thousand genes in the human genome, which might encode, by some estimation, 100,000 proteins as a result of alternative splicing. To fully understand the biological functions and functional mechanisms of these proteins, knowledge of their 3-D structures is required. The ambitious Structural Genomics Initiatives, launched by NIH (National Institutes of Health) in 1999, intends to solve these protein structures within the next ten years, through the development and application of significantly improved experimental and computational technologies.

A protein structure is typically solved using x-ray crystallography or nuclear magnetic resonance spectroscopy (NMR), which are costly and very time-consuming (ranging from months to years per structure) and is quite difficult for high-throughput production. The overall strategy of the NIH Structural Genomics Initiatives is to solve protein structures using experimental techniques like x-ray crystallography or NMR only for a small fraction of all the proteins and to employ computational techniques to model the structures for the rest of the proteins. The basic premise is that though there could be millions of proteins in nature, the number of unique structural folds is probably 2-3 (or even more) orders of magnitude smaller.

Hence, by strategically selecting the proteins with unique structural folds for experimental solutions, we can put the vast majority of other proteins “within the modeling distance” of these proteins. Model-based structure prediction techniques could play a significant role in helping to achieve the goal of the Structural Genomics Initiatives. Protein threading represents one such prediction technique.

Protein threading can be used for both structure prediction and protein fold recognition, i.e., detection of homologous proteins. Numerous computer algorithms have been proposed for protein structure prediction, based on the threading approach. Based on the energy function models and computational methods, they can be grouped into three classes:

1. models in which the energy function does not include the pairwise interaction preferences explicitly. For this kind of model, a simple dynamic programming is employed to optimize the energy function. GenTHREADER is a typical example (see D. T. Jones. J. Mol. Biol., 287:797-815, 1999). The prediction speed is fast, but theoretically, the prediction accuracy is worse than those incorporating pairwise interactions;

2. models in which the energy function includes the pairwise interaction preferences. However, it has been proved that this problem is NP-hard when variable gaps and pairwise interactions are considered simultaneously (see R. H. Lathrop. Protein Engineering, 7:1059-1068, 1994). Various kinds of approximation algorithms are used to optimize the energy function, including double dynamic programming (see D. T. Jones, W. R. Taylor, and J. M. Thornton. Nature, 358:86-98, 1992), frozen approximation (see A. Godzik, A. Kolinski, and J. Skolnick. Journal of Molecular Biology, 227:227-238, 1992), and Monte Carlo sampling algorithm (see S. Bryant. Proteins: Struct. Funct. Genet., 26:172-185, 1996). Unfortunately, T. Akutsu et al. have proved that this problem is MAX-SNP-hard (see T. Akutsu and S. Miyano. Theoretical Computer Science, 210:261-275, 1999), which means that it cannot be approximated to arbitrary accuracy in polynomial time; and

3. models in which the energy function includes the pairwise interaction preferences and an exact algorithm is designed to optimize the energy function. Xu et al. have proposed a divide-and-conquer method (see Y. Xu et al. Journal of Computational Biology, 5(3):597-614, 1998) which runs fast on simple protein template (interaction) topology but could take a long time for proteins with dense residue-residue interactions. In addition, this approach does not treat the following two special features explicitly:

a. interaction significance could be different from residues to residues; and

b. interaction potentials could be heavily correlated with other non-pairwise scores such as mutation scores and fitness scores.

There is therefore a need for an improved method and system of protein structure analysis, provided with consideration for the problems outlined above.

### SUMMARY OF THE INVENTION

It is therefore an object of the invention to provide a novel method and system of protein analysis which obviates or mitigates at least one of the disadvantages of the prior art.

The main focus in this patent application is on the development of a globally optimal and practically efficient threading algorithm based on an alignment model incorporating pairwise interaction preferences explicitly, and allowing variable gaps by using an integer programming approach. Integer programming formulation can fully exploit the abovementioned special features of the pairwise interaction preferences. It allows one to use the existing powerful linear programming packages together with a branch and bound algorithm to rapidly arrive at the optimal alignment. This is the first time that integer programming and linear programming has been applied to protein threading.

One aspect of the invention is broadly defined as a method of aligning a query protein sequence with a template consisting of a set of pre-selected protein structures in a database, comprising the steps of: selecting an energy function, the energy function being a sum of energy parameters and weighting factors; determining values for weighting factors in the energy function; establishing linear programming (LP) constraints for threading (or aligning) the query protein sequence with each structure in the set of pre-selected protein structures in a database; and performing a linear programming analysis based on a linear programming formulation including the energy function under the constraints, to optimally align the query protein with the template.

Another aspect of the invention is defined as A method of alignment comprising the steps of: formulating the protein threading problem as a large scale integer programming problem; relaxing this problem to a linear programming problem; and solving the integer program by a branch-and-bound method.

A further aspect of the invention is defined as a system for aligning proteins comprising: a computer operable to align a query protein sequence with a template consisting of a set of pre-selected protein structures in a database, by performing the steps of: selecting an energy function; determining values for weighting factors in the energy function; establishing linear programming (LP) constraints for threading (or aligning) the query protein sequence with each structure in the set of pre-selected protein structures in a database; and performing a linear programming analysis based on a linear programming formulation including the energy function under the constraints, to optimally align the query protein with the template.

### BRIEF DESCRIPTION OF THE DRAWINGS

These and other features of the invention will become more apparent from the following description in which reference is made to the appended drawings in which:

FIG. 1 presents a block diagram of a personal computer (PC) which may be used in an embodiment of the invention;

FIG. 2 presents a flow chart of a broad method of the invention;

FIG. 3 presents a graph of computing time vs. sequence size for threading 100 sequences to template 119I;

FIG. 4 presents a template contact graph;

FIG. 5 presents a compressed contact graph in an embodiment of the invention; and

FIG. 6 presents a flow chart of an exemplary method of the invention.

### DETAILED DESCRIPTION OF PREFERRED EMBODIMENTS OF THE INVENTION

1.0 Introduction

The process of protein three-dimensional structure prediction through threading has been extensively studied and various models and algorithms have been proposed. To improve accuracy and efficiency of the threading process, this invention proposes a new method: protein threading via linear programming. Based on the contact map model of protein 3D structure, we formulate the protein threading problem as a large scale integer programming problem, then relax to a linear programming problem, and finally solve the integer program by a branch-and-bound method. The final solution is optimal with respect to energy functions incorporating pairwise interaction and allowing variable gaps. Our formulation generally allows the linear program to provide integral solutions. The algorithm has been implemented as a software package named RAPTOR (RApid Protein Threading predictOR). Experimental results show that RAPTOR significantly outperforms other programs for fold recognition.

2.0 Model

Before describing the invention, a review of the notation and framework for the discussion will be presented.

We represent an amino acid sequence, of length m, of a protein template by t₁ t₂ . . . tₘ and the query sequence, of length n, by s₁ s₂ . . . sₙ. In formulating the protein threading problem, we follow a few basic assumptions widely accepted by the protein threading community (see Y. Xu et al. Journal of Computational Biology, 5(3):597-614, 1998). We assume that:

1. each template consists of a linear series of cores with the connecting loops between the adjacent cores. Each core is a conserved segment of an α-helix or β-sheet secondary structure among the protein's homologs. Although the secondary structure is often conserved, insertion or deletion may occur within a secondary structure. So we only keep the most conserved part. Let cᵢ=core (headᵢ, tailᵢ) denote all cores of one template, where i=1, 2, . . . , M with M being the number of the cores, and 1≦head₁≦tail₁<head₂≦tail₂< . . . <head_{M}≦tail_{M}≦m. The region between tailᵢ and headᵢ₊₁ is a loop. The length of cᵢ is lenᵢ=tailᵢ−headᵢ+1. Let locᵢ denote the sum of the length of all cores before cᵢ, that is,

$${loc}_{i} = {\sum }_{j = 1}^{i - 1} {len}_{j} ;$$

2. when aligning a query protein sequence with a template, alignment gaps are confined to loops, i.e., the regions between cores or the two ends of the template. The biological justification is that cores are conserved so that the chance of insertion or deletion within them is very little; and

3. we consider only interactions between core residues. It is generally believed that interactions involving loop residues can be ignored as their contribution to fold recognition is relatively insignificant. We say that an interaction exists between two residues if the spatial distance between their Cα atoms is within 7 A and they are at least 4 positions apart in the template sequence. We say that an interaction exists between two cores if there exists at least one residue-residue interaction between the two cores.

Our threading energy function consists of an environment fitness score Eₛ, mutation score Eₘ, secondary structure compatibility score Eₛₛ, gap penalty E_{g} and pairwise interaction score Eₚ. The overall energy function E has the following form:

E=WₘEₘ+WₛEₛ+WₚEₚ+W_{g}E_{g}+Wₛₛ Eₛₛ,

where Wₘ, Wₛ, Wₚ, W_{g} and Wₛₛ are weight factors to be determined by training.

Global alignment and global-local alignment methods are employed to align one template to one sequence. For a detailed description of the alignment process, refer to Fischer's paper (see D. Fischer et al. pages 300-318. PSB96, 1996). In the case that the template size is larger than the query sequence size, it is possible that some cores at the two ends of the template cannot be aligned to the sequence. But we can always extend the sequence by adding some “artificial” amino acids to the two ends of the sequence to make all cores are aligned to the (extended) sequence. All scores involving those extended positions are set to be 0.

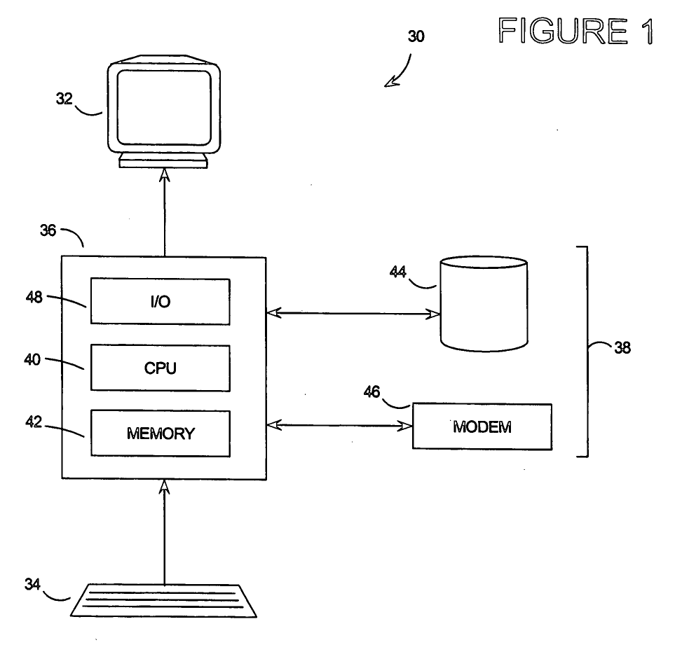

The method of the invention may be applied on virtually any computer or microprocessor-based system. A server, minicomputer or mainframe on a local area network or connected to the Internet, could, for example execute the algorithm and pass the results of any queries back to the user. An exemplary system on which the invention may be implemented, is presented as a block diagram in FIG. 1.

This computer system 30 includes a display 32, keyboard 34, computer 36 and external devices 38. The computer 36 may contain one or more processors or microprocessors, such as a central processing unit (CPU) 40. The CPU 40 performs arithmetic calculations and control functions to execute software stored in an internal memory 42, preferably random access memory (RAM) and/or read only memory (ROM), and possibly additional memory 44. The additional memory 44 may include, for example, mass memory storage, hard disk drives, floppy disk drives, magnetic tape drives, compact disk drives, program cartridges and cartridge interfaces such as those found in video game devices, removable memory chips such as EPROM or PROM, or similar storage media as known in the art. This additional memory 44 may be physically internal to the computer 36, or external as shown in FIG. 1.

The computer system 30 will also generally include a communications interface 46 which allows software and data to be transferred between the computer system 30 and external systems. Examples of communications interface 46 can include a modem, a network interface such as an Ethernet card, or a serial or parallel communications port. Software and data transferred via communications interface 46 are in the form of signals which can be electronic, electromagnetic, optical or other signals capable of being received by communications interface 46. Multiple interfaces, of course, can be provided on a single computer system 30.

Input and output to and from the computer 36 is administered by the input/output (I/O) interface 48. This I/O interface 48 administers control of the display 32, keyboard 34, external devices 38 and other such components of the computer system 30.

The invention is described in these terms for convenience purposes only. It would be clear to one skilled in the art that the invention may be applied to other computer or control systems 30.

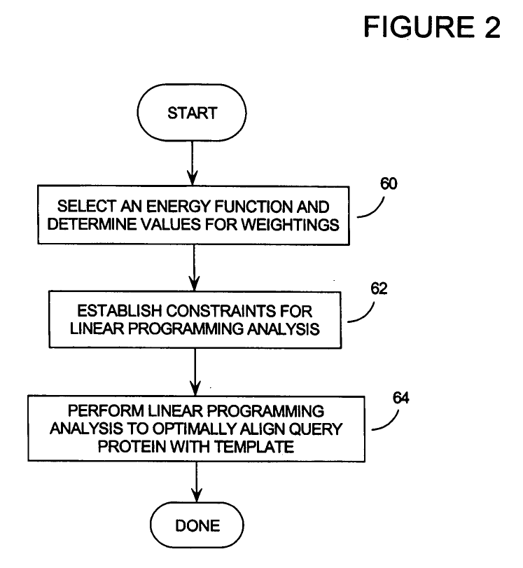

The invention can be generally represented per the flow chart of FIG. 2. Briefly, this figure presents a method of aligning a query protein with a template which proceeds as follows:

First, an energy function is selected, and appropriate weighting factors determined, per step 60. Energy functions and various methods for determining weighting factors are known in the art. It is preferable though, that the energy function described above, be used.

Next, linear programming constraints are established for threading (or aligning) the query protein sequence to each of pre-selected protein structures in the database, per step 62. A detailed discussion follows on the various constraints that may be used. Clearly though, the invention does not turn on any particular set of constraints being employed.

Finally, a linear programming analysis based on the LP formulation generated at step 62, is performed at step 64. This LP analysis considers the energy function under the constraints, in an attempt to optimally align the query protein with the template.

This technique has been shown to result in a significant advance over prior methods.

3.0 Formulation

Definition 3.1 We use an undirected graph CMG=(V, E) to denote the contact map graph of a protein template structure. Here, V={c₁, c₂, . . . , cₘ} where cᵢ represents the i^{th} core, and E={(cᵢ, cⱼ)| there is an interaction between cᵢ and cⱼ, or |i−j|=1}.

For simplicity, when we say that core cᵢ is aligned to position so we always mean that core cᵢ=(headᵢ, tailᵢ) is aligned to segment (sⱼ,sⱼ₊ₗₑₙ_{ᵢ}₋₁) In order to speed up the search, RAPTOR employs the knowledge-based altering process proposed in PROSPECT (again, see Y. Xu et al. Journal of Computational Biology, 5(3):597-614, 1998) which indicates certain alignments as invalid.

Definition 3.2 Let B denote the alignment bipartite graph of one threading pair. Each core of the template corresponds to one vertex in B, labelled as cᵢ(i=1, 2, . . . , M), each residue in the query sequence corresponds to one vertex in B, labelled as sⱼ(j=1, 2, . . . n). The edges of B consist of all valid alignments (after initial filtering) between each core and each sequence position. The edges of B are also called the alignment edges.

Definition 3.3 For any two different edges e₁=(cᵢ₁, sⱼ₁) and e₂=(cᵢ₂, sⱼ₂) in an alignment bipartite graph B, if (locᵢ₁−locᵢ₂)×(sⱼ₁+locᵢ₂−locᵢ₁−sⱼ₂)≦0, then we say e₁ and e₂ are in conflict.

The proof of the following three lemmas is omitted due to space limitations.

Lemma 3.1 For any three different edges eᵣ=(cᵢₙ sⱼᵣ), r=1, 2, 3 and locᵢ₁<locᵢ₂<locᵢ₃, if e₁ conflicts with e₂ and e₂ conflicts with e₃, then e₁ conflicts with e₃.

Lemma 3.2 For any three different edges eᵣ=(cᵢₙ sⱼᵣ), r=1, 2, 3 and locᵢ₁<locᵢ₂<locᵢ₃, if e₁ does not conflict with e₂ and e₂ does not conflict with e₃, then e₁ does not conflict with e₃.

Lemma 3.3 For any three different edges eᵣ=(cᵢₙ sⱼᵣ), r=1, 2, 3 and locᵢ₁<locᵢ₂<locᵢ₃, if e₁ conflicts with e₃, then e₂ conflicts with e₃ or e₂ conflicts with e₁.

Definition 3.4 An alignment is called a valid alignment if:

1. each core of the template is aligned to some position of the (extended) sequence. As mentioned before, global and global-local alignment are employed; and

2. for any two different cores c₁ and c₂, their two alignment edges do not conflict in the alignment graph.

Let x_{i,l}, be a boolean variable such that x_{i,l}=1 if and only if core cₗ is aligned to position sₗ. Similarly, for any (cₗ₁, cₗ₂) ε E (C M G), let y_{(l1, l1), (l2, l2)} indicate the pairwise interactions between x_{l1,l1,} and x_{l2,l2} if the two edges (cₗ₁, sₗ₁), (cₗ₂, sₗ₂) do not conflict. y_{(l1, l1), (l2, l2)}=1 if and only if x_{l1,l1}=1 and x_{l2,l2}=1. We say y_{(l1, l1), (l2, l2)} is generated by x_{l1, l1} and x_{l2, l2}.

The x variables are called the alignment variables and y variables are called the interaction variables. Let D [i] denote all valid query sequence positions that cₗ could be aligned to. Let R [i, j, l] denote all valid alignment positions of cⱼ given cₗ is aligned to sₗ.

Now the objective function of the protein threading problem can be formulated as follows:

min WₘEₘ+WₛEₛ+WₚEₚ+W_{g}E_{g}+WₛₛEₛₛ; (1)

$$\begin{matrix}{E}_{m} = {\sum }_{i = 1}^{M} {\sum }_{l ∈ D [ i ]} [ {x}_{i , l} \times {\sum }_{r = 0}^{leni - 1} Mutation ( {head}_{i} + r , l + r ) ] ; & ( 2 ) \\ {E}_{s} = {\sum }_{i = 1}^{M} {\sum }_{l ∈ D [ i ]} [ {x}_{i , l} \times {\sum }_{r = 0}^{leni - 1} Fitness ( {head}_{i} + r , j + r ) ] ; & ( 3 ) \\ {E}_{ss} = {\sum }_{i = 1}^{M} {\sum }_{l ∈ D [ i ]} [ {x}_{i , l} \times {\sum }_{r = 0}^{leni - 1} SS ( {t}_{headi + r} , {S}_{j + r} ) ] ; & ( 4 ) \\ {E}_{p} = {\sum }_{1 \leq i < j \leq M , ( c i , c j ) ∈ E ( CMG )} {\sum }_{l ∈ D [ i ]} \times {\sum }_{k ∈ R [ i , j , l ]} {y}_{( i , l ) , ( j , k )} P ( i , j , l , k ) ; & ( 5 ) \\ P ( i , j , l , k ) = {\sum }_{u = 0}^{leni - 1} {\sum }_{v = 0}^{lenj - 1} {δ}_{} ( {t}_{headi + u} , {t}_{headj + v} ) Pair ( l + u , k + v ) ; & ( 6 ) \\ {E}_{g} = {\sum }_{i = 1}^{M} {\sum }_{l ∈ D [ i ]} {\sum }_{k ∈ R [ i , i + 1 , l ]} {y}_{( i , l ) , ( i + 1 , k )} G ( i , l , k ) ; & ( 7 )\end{matrix}$$

where δ=(tᵤ, tᵥ)=1 if there is an interaction between residues at position u and v in the template, otherwise δ=0. G(i, l, k) is the gap potential between cₗ and cₗ₊₁, when they are aligned to query sequence position l and k respectively. G(i, l, k) could be computed by dynamic programming in advance given i, l, and k.

The constraint set is as follows:

$$\begin{matrix}{\sum }_{j ∈ D [ i ]} {x}_{i , j} = 1 , i = 1 , 2 , … , M ; & ( 8 ) \\ {\sum }_{l \geq {l}_{0} , l ∈ D [ i ]} {x}_{i , l} + {\sum }_{k ∈ D [ i + 1 ] - R [ i , i + 1 , {l}_{0} ]} {x}_{i + 1 , k} \leq 1 , {l}_{0} ∈ D [ i ] , i = 1 , 2 , … , M - 1 ; & ( 9 ) \\ {\sum }_{k ∈ R [ i , j , l ]} {y}_{( i , l ) , ( j , k )} \leq {x}_{i , l} , ∀ l ∈ D [ i ] , i , j = 1 , 2 , … , M ; & ( 10 ) \\ {\sum }_{l ∈ R [ j , i , k ]} {y}_{( i , l ) , ( j , k )} \leq {x}_{j , k} , ∀ k ∈ D [ j ] , i , j = 1 , 2 , … , M ; & ( 11 ) \\ {\sum }_{k ∈ R [ i , j , l ]} {y}_{( i , l ) , ( j , k )} \geq {x}_{i , l} + {\sum }_{k ∈ R [ i , j , l ]} {x}_{j , k} - 1 , l ∈ D [ i ] , i , j = 1 , 2 , … , M ; & ( 12 ) \\ {\sum }_{l ∈ R [ j , i , k ]} {y}_{( i , l ) , ( j , k )} \geq {x}_{j , k} + {\sum }_{l ∈ R [ j , i , k ]} {x}_{i , l} - 1 , k ∈ D [ j ] , i , j = 1 , 2 , … , M ; & ( 13 )\end{matrix}$$

xᵢⱼ≧0,j ε D[i], i=1, 2, . . . , M; (14)

y_{(i,l)(j,k)}≧0, ∀l ε D[i], k ε D[i], i,j=1, 2, . . . , M. (15)

Constraint 8 says that one core can be aligned to a unique sequence position. Constraint 9 forbids the conflicts between the adjacent two cores. Therefore, based on lemma 3.2, this constraint can guarantee that there are no conflicts between any two cores if variable x and y are integral. Constraints 10 and 11 say that at most one interaction variable can be 1 between any two cores that have interactions between each other. Constraints 12 and 13 enforce that if two cores have their alignments to the sequence respectively and also have interactions between them, then at least one interaction variable should be 1. Constraints 8, 14 and 15 guarantee x and y to be between 0 and 1 when this problem is relaxed to a linear program.

There is another set of constraints which can replace Constraints 9-13. They are:

x_{i,l}+x_{i+l,k}≦1, ∀k ε D[i+1]−R[i,i+1,l]; (16)

y_{(i,l)(j,k)}≦x_{i,l}, k ε R[i, j, l], (cᵢ, cⱼ) ε E(CMG); (17)

y_{(i,l)(j,k)}≦x_{j,k}, l ε R[j, i, k], (cᵢ, cⱼ) ε E(CMG); (18)

y_{(i,l)(j,k)}≧x_{i,l}+x_{j,k}−1, (cᵢ, cⱼ) ε E(CMG); (19)

Constraint 16 forbids the conflict between two neighbouring cores and Constraints 17-19 guarantee that one interaction variable is I if and only if its two generating x variables are 1. Constraints 16-19 can be inferred from Constrains 9-13, but the converse is not true. Therefore, Constraints 16-19 are weaker than Constraints 9-13.

In order to improve running time, we found yet another set of Constraints 20 and 21 from which both 9-13 and 16-19 can be inferred (proofs omitted due to space limitations):

$$\begin{matrix}{\sum }_{k ∈ R [ i , j , l ]} {y}_{( i , l ) , ( j , k )} = {x}_{i , l} , ( {c}_{i} , {c}_{j} ) ∈ E ( CMG ) ; & ( 20 ) \\ {\sum }_{l ∈ R [ j , i , k ]} {y}_{( i , l ) , ( j , k )} = {x}_{j , k} , ( {c}_{i} , {c}_{j} ) ∈ E ( CMG ) ; & ( 21 )\end{matrix}$$

Constraints 20 and 21 imply that one x variable is 1 is equivalent to that one of the y variables generated by it is 1. These two are the strongest constraints. Experimental results show that our algorithm with Constraints 20 and 21 (combining with Constraints 8, 14 and 15) runs significantly faster. The RAPTOR program uses Constraints 20 and 21 by default.

4.0 RAPTOR Implementation

4.1 Scoring System

We calculated the averaged energy over a set of homologous sequences, as demonstrated in PROSPECT-II (see D. Kim, D. Xu, J. Guo, K. Ellrott, and Y. Xu. 2002. Manuscript.). Given a query sequence of length n, an n×20 frequency matrix PSFM (Position Specific Frequency Matrix) is calculated by using PSI-BLAST (see S. F. Altschul et al. Nucleic Acids Research, 25:3389-3402, 1997) with a maximum iteration number being set to 5. Each column of this matrix describes the occurring frequency of 20 amino acids at this position. Assume a template position i is aligned to the sequence position j. Then the mutation score and fitness score are calculated as follows:

$$Mutation ( i , j ) = {\sum }_{a} {p}_{j , a} M ( {t}_{i} , a )$$

$$Fitness ( i , j ) = {\sum }_{a} {p}_{j , a} F ( {env}_{i} , a )$$

where p_{j,a} represents the occurring frequency of amino acid a at sequence position j, M (a, b) represents the mutation potential between two amino acids a and b which is taken from PAM250 matrix (see R. M. Schwartz and M. O. Dayhoff, pages 353-358. Natl. Biomed. Res. Found., 1978), and F (env, a) denote the fitness potential when amino acid a is placed into environment env.

The nine combinations of three secondary structure types (α-helix, β-strand and coil) and three solvent accessibility levels are used to define the local environments of a position in the template. The boundaries between the three solvent accessibility levels are at 7% and 37%. Secondary structure and solvent accessibility assignments are all taken from FSSP database (see L. Holm and C. Sander. Science, 273:595-602, 1996).

The gap penalty function is assumed to be an affine function, i.e., a gap open penalty plus a length-dependent gap extension penalty. The gap open penalty is set at 10.6 and gap elongation penalty is 0.8 per single gap (see G. H. Gonnet et al. Science, 256:1443-1445,1992). We use PSIPRED (see D. T. Jones. J. Mol. Biol., 292:195-202, 1999) to predict the secondary structure of the query sequence. If the two ends of an interaction are aligned to j₁^{th} and j₂^{th} positions of the query sequence respectively, then the pair score for this interaction is given by:

$$Pair ( {j}_{1} , {j}_{2} ) = {\sum }_{a} {p}_{j 1 , a} {\sum }_{b} {p}_{j2 , b} P ( a , b )$$

where P (a, b) denotes the pairwise interaction potential between two amino acids a and b. F, Pare taken from PROSPECT-II (again, see D. Kim, D. Xu, J. Guo, K. Ellrott, and Y. Xu. 2002. Manuscript).

4.2 Branch-and-Bound Method

We use a branch-and-bound algorithm to solve the above integer programming problem. First we relax the above integer program by allowing all x and y to be real between 0 and 1 and solve the resulting linear program. If the solution (x*, y*) of the linear program is integral, then we get the optimal solution. Otherwise, we select one non-integral variable according to some criterion, and generate two subproblems by setting it to 0 and 1 respectively.

These two subproblems are solved recursively. More details on solving the integer programming problem can be found in Laurence A. Wolsey, John Wiley and Sons, Inc., 1998. The IBM OSL(Optimization and Solution Library) package is used to implement this process.

4.3 Weight Training

The weight factors Wₘ, Wₛ, Wₚ, W_{g}, Wₛₛ are chosen through optimizing the overall alignment accuracy. The optimal alignment accuracy does not necessarily imply the best fold recognition capability though. In the following subsection, an SVM (Support Vector Machine) method is used to carry out fold recognition. A set of 95 structurally-aligned protein pairs are chosen from Holm's test set (see L. Holm and C. Sander. ISMB, 5:140-146, 1997) as the training samples, each of which only has fold-level similarity. The alignments generated by RAPTOR is compared with the structural alignments generated by SARF (see N. N. Alexandrov. Protein Engineering, 9:727-732, 1996). An alignment for a residue is regarded as correct if it is within 4 residue shift away from the correct structure-structure alignment by SARF. The overall alignment accuracy is defined as the ratio between the number of the correctly-aligned positions of all threading pairs and the number of the maximum alignable positions.

Our objective is to maximize the overall alignment accuracy. A genetic algorithm plus a local pattern search method implemented in DAKOTA (see M. S. Eldred et al. Technical Report SAND2001-3796, Sandia, 2002) is used to search for the optimal weight factors. We attained 56% alignment accuracy over this set of training pairs. A set of 1100 protein pairs which are in the fold-level similarity is also generated from Holm's test set (again, see L. Holm and C. Sander. ISMB, 5:140-146, 1997) to test the weight factors and 50% alignment accuracy is attained. We have also selected 95 structurally-aligned protein pairs from Holm's test set, each of which is in superfamily-level or family-level similarity, 80% alignment accuracy is achieved when the same set of weight factors is used.

4.4 Z-Score and Fold Recognition

After threading one pair of sequence and template, z-score is calculated according to the method proposed by S. H. Bryant and S. F. Altschul. in Curr. Opin. Struct. Biol., 5:236-244, 1995, to cancel out the composition bias. Let z_{raw} denote this kind of z-score. However, since the accurate z_{raw} is expensive to compute, we just approximate it by:

1. fixing the alignment positions;

2. shuffling the query sequence randomly; and

3. calculating the alignment scores based on the existing alignment rather than doing optimal alignments again and again.

The free software SVM light (see T. Joachims. MIT Press, 1999) with RBF kernel is employed to adjust the approximate z-score. The reader may also refer to Vapnik's book (see V. N. Vapnik. Springer, 1995) for a comprehensive tutorial of SVM.

A set of 60000 training pairs formed by all-against-all threading between 300 templates (randomly chosen from the FSSP database) and 200 sequences (randomly chosen from Holm's test set—again, see L. Holm and C. Sander. ISMB, 5:140-146, 1997) is used as the training samples of our SVM model. The relationship between two proteins is judged based on SCOP database (see A. G. Muzrin et al. J. Mol. Biol., 247:536-540, 1995). If one pair is in at least fold-level similarity, then it is treated as a positive example, otherwise a negative example. Each of training samples consists of the following features:

1. z_{raw};

2. the sequence size;

3. the template size;

4. the number of cores in the template;

5. the sum of the core size in the template;

6. the number of aligned cores;

7. the number of aligned positions;

8. the number of identical residues;

9. the number of contacts with both ends on the aligned cores;

10. the number of cut contacts with one end on the aligned cores and the other on the unaligned cores;

11. the total score;

12. mutation score;

13. singleton fitness score;

14. gap score;

15. secondary score;

16. pair score.

Given one threading result, SVM outputs a real value. The value greater than 0 means this threading pair is in at least fold-level similarity. We do not use this directly due the abundance of the false negatives. We calculate the final z-score for each query sequence. For all threading pairs of one given sequence, let o₁, o₂, . . . o_{q} denote the outputs from SVM model. The final z-score is calculate by [oₗ−u (o)]/[std (o)], where u(o) is the mean value of oₗ and std (o) is the standard deviation of oₗ. Daniel Fischer's benchmark (see D. Fischer et al. pages 300-318. PSB96, 1996) is used to fix the parameters of the model.

5 Preliminary Experimental Results

Fischer's benchmark consists of 68 target sequences and 301 templates. RAPTOR ranks 56 pairs out of 68 pairs as top 1, achieving an 82% prediction rate, while the previous best was 76.5%.

The fold recognition performance of RAPTOR was further tested on Lindahl's benchmark set consisting of 976 protein sequences (see E. Lindahl and A. Elofsson. J. Mol. Biol., 295:613-625, 2000). By threading them all against all, there are totally 976×975 pairs. We measure RAPTOR's performance in three different similarity levels: fold, superfamily and family. The results are shown in Table 1. The results of other methods are taken from Shi et al.'s paper (see J. Shi, L. B. Tom, and M. Kenji. J. Mol. Biol., 310:243-257, 2001).

TABLE 1
The performance of RAPTOR at three different similarity levels

| | | | Super- | | | |
|---|---|---|---|---|---|---|
| | Family | Family | family | Superfamily | Fold | Fold |
| Method | Top 1 | Top 5 | Top 1 | Top 5 | Top 1 | Top 5 |
| RAPTOR | 75.2 | 77.8 | 39.3 | 50 | 25.4 | 45.1 |
| RAPTOR-np | 68.9 | 72.8 | 34 | 49.7 | 19 | 36.6 |
| FUGUE | 82.2 | 85.8 | 41.9 | 53.2 | 12.5 | 26.8 |
| PSI-BLAST | 71.2 | 72.3 | 27.4 | 27.9 | 4 | 4.7 |
| HMMER-PSI | 67.7 | 73.5 | 20.7 | 31.3 | 4.4 | 14.6 |
| BLAST | | | | | | |
| SAMT98-PSI | 70.1 | 75.4 | 28.3 | 38.9 | 3.4 | 18.7 |
| BLAST | | | | | | |
| BLASTLINK | 74.6 | 78.9 | 29.3 | 40.6 | 6.9 | 16.5 |
| SSEARCH | 68.6 | 75.7 | 20.7 | 32.5 | 5.6 | 15.6 |
| THREADER | 49.2 | 58.9 | 10.8 | 24.7 | 14.6 | 37.7 |

As shown in Table 1, the performance of RAPTOR at the fold level is much better than other analysis software. At the superfamily level, RAPTOR performs a little bit worse than FUGUE (again, see J. Shi, L. B. Tom, and M. Kenji. J. Mol. Biol., 310:243-257, 2001), the best method (for superfamily and family level) listed in this table. However, at the family level, RAPTOR performs better than only THREADER, which means that RAPTOR is superior in recognizing fold-level similarity but poor in doing homology detection. RAPTOR-np is a variant of RAPTOR without considering pairwise interactions when doing optimal alignment, but the pairwise score is still calculated based on the non-pairwise alignment. The corresponding weight factors and SVM model are optimized separately using the same sets of training samples. Compared with RAPTOR-np, RAPTOR is better in fold level and superfamily level and same in family level. Thus, we may conclude that a strict treatment of the pairwise interactions is necessary for fold level recognition or even superfamily level.

6 Computing Efficiency Issues

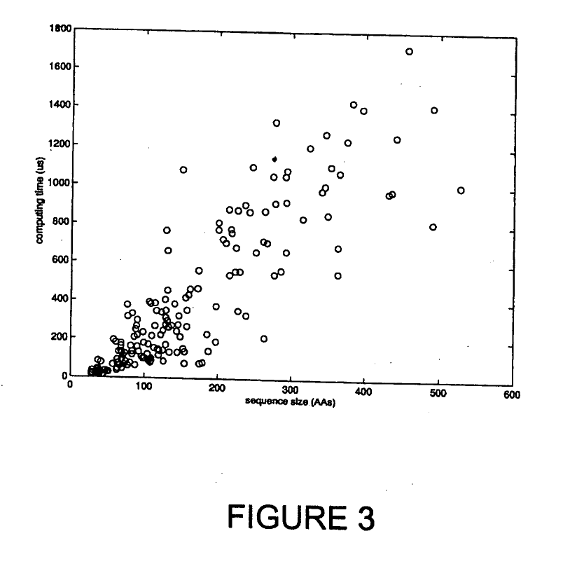

An outstanding advantage of the invention is that the memory requirement is just about O (M²n²) and, generally the computing time does not increase exponentially with respect to the sequence size. FIG. 3 presents a graph of the CPU time of threading 100 sequences (chosen randomly from Lindahl's benchmark) with size ranging from 25 to 572 to a typical template 1191 of length 162 (with topological complexity 3 and 12 cores; see Y. Xu et al. Journal of Computational Biology, 5(3):597-614, 1998). According to Xu et al., the computing time of PROSPECT is O (Mn⁵) and its memory usage is O (Mn⁴). The observed memory usage of RAPTOR is 100˜200M for most of threading pairs. FIG. 3 shows that the computing time of our algorithm increases very slowly with respect to the sequence size. In fact, we found out that our relaxed linear programming gave the integral solutions most of time or generated only a few branch nodes when the solution was not integral.

7 Improving LP Efficiency

Protein threading is a very effective method to do fold recognition. Profile-based method runs very fast but could only recognize easy targets (Homology Modelling targets). Pairwise contact potential plays an important role in recognizing hard targets (Fold Recognition targets). However, if pairwise contacts are considered and variable gaps are allowed, then protein threading problem is NP-hard. Therefore, an efficient and exact algorithm is indispensable for protein threading in order to do fold-level recognition. PROSPECT makes use of a clever divide-and-conquer algorithm to search for the globally optimal alignment between templates and sequences. Its computational complexity depends on the topological complexity of the template contact graph. It is very efficient for those templates whose contact graphs have low topological complexity. PROSPECT could effectively exploit the sparseness of contact graphs. If the distance cutoff is 7 A, there are about three quarters of contact graphs which have low complexity (≦4).

However, PROSPECT could not deal with those templates with high topological complexity. The integer programming method could be used to deal with those templates with high complexity by exploiting the relationship between various kinds of scores contained in the energy function. For a long sequence and a complex contact graph, there are often tens of millions of variables involved in the integer program. Even for a topologically complex contact graph, there is often some regular part contained in this graph, i.e., this graph might have a subgraph which is topologically simple. The integer programming approach could not automatically take advantage of the partial regularity of the contact graph. We could use a divide-and-conquer algorithm to align this kind of regular subgraph to the sequence first, combining the integer programming approach and the divide-and-conquer algorithm. Before employing the powerful integer programming approach to formulate the problem, we first compress the contact graph to generate a dense contact graph by some graph reduction operation. During the process of reduction, simple dynamic programming and divide-and-conquer algorithm could be used to align one segment to the sequence.

7.1 HyperGraph-Based Integer Program Formulation

In this section, we will present a hypergraph-based integer program formulation which could be regarded as the generalization of the integer program formulation mentioned in the above sections. This kind of formulation could be used in two aspects. First, if the threading energy function takes the multiple-wise rather than pairwise contact potential into account, then the template contact graph becomes the hypergraph. We need to deal with a hypergraph based threading problem directly. Second, even if only the pairwise contact potential is considered, after graph reduction operation which would be discussed in details in the following sections, the reduced contact graph could become a hypergraph. In order to take advantage of the integer programming approach, we need to generate our simple graph-based integer program formulation to the hypergraph-based formulation.

Definition 7.1 We use an undirected contact hypergraph HCMG=(V, HE) to model the contact map graph of a protein template structure, V={c₁, c₂ . . . c_{M}), where cᵢ denotes the i^{th} core and H E={e=(cᵢ₁, cᵢ₂, . . . , cᵢₖ)| there is one multiple-wise interaction among cᵢ₁, cᵢ₂, . . . , cᵢₖ.{

The only difference between the hypergraph-based threading problem and the simple graph based threading problem is that the energy function of hypergraph-based threading consists of the multiple-wise interaction preferences. If there is a multiple-wise interaction among t₁, t₂, . . . , tₖ, Let P(s₁, s₂, . . . , sₖ, t₁, t₂, . . . , tₖ) denote the multiple-wise interaction score when aligning sₗ to template position tₗ, then the objective of hypergraph-based threading is to minimize

ΣF(sᵢ,tᵢ)+ΣP(s₁, s₂, . . . , sₖ, t₁, t₂, . . . tₖ).

For any e=(cᵢ₁, cᵢ₂, . . . , cᵢₖ) ε H E, let y_{e, i1, i2, . . . , ik} indicate the multiple-wise interaction among x variable x_{i1, i1}, x_{i2, i2}, . . . , x_{ik,ik}·y_{e, i1, i2, . . . ik}=1 if and only if core cᵢᵣ is aligned to sequence position lᵣ (r=1, 2, . . . , k). Then we could formulate the objective function of the hypergraph based threading as follows:

MᵢₙΣx_{ir, lr}F(lᵣ,iᵣ)+Σy_{(e, l1, l2, . . . , lk)}P_{l1, l2, . . . ,lk, e}

where F (lₙiᵣ) denote the single-wise score when core iᵣ is aligned to sequence position lᵣ and P_{(l1, l2, . . . , lk, e)} the multiple-wise interaction score when e=(cᵢ₁, cᵢ₂, . . . , cᵢₖ) and core cᵢᵣ is aligned to sequence position lᵣ.

Let D [i] denote the set of sequence positions that core cᵢ could be aligned to and R [i, j, l] the set of sequence positions that core cⱼ could be aligned to given that core cₗ is aligned to sequence position l.

The constraint set is as follows:

$$\begin{matrix}{\sum }_{l ∈ D [ i ]} {x}_{i , l} = 1 & ( 22 ) \\ For any e = ( {c}_{{i}_{1}} , {c}_{{i}_{2}} , … , {c}_{{i}_{k}} ) ∈ H E , ∀ {l}_{r} ∈ D [ {i}_{r} ] , we have \\ {x}_{{i}_{r}} , {l}_{r} = {\sum }_{{l}_{p} ∈ R [ {i}_{r} , {i}_{p} , {l}_{r} ]} y ( e , {l}_{1} , {l}_{2} , … , {l}_{k} ) & ( 23 )\end{matrix}$$

y_{e,l}_{₁}_{,l}_{₂}_{, . . . ,l}ₖ ε {0, 1} (24)

x_{i,l}_{ᵢ} ε {0, 1} (25)

7.2 Contact Graph Reduction

For a long protein template and a long query sequence, the number of the integer variables is often huge, so it will take the integer program a very long time to find the optimal solution from a search space of exponential size. Before applying the integer programming approach to the threading problem, we try to decrease the number of the integer variables by employing a graph reduction technique onto the template contact graph.

This technique is effective because many template contact graphs are sparse or contain sparse subgraphs.

7.2.1 Graph Reduction Operation

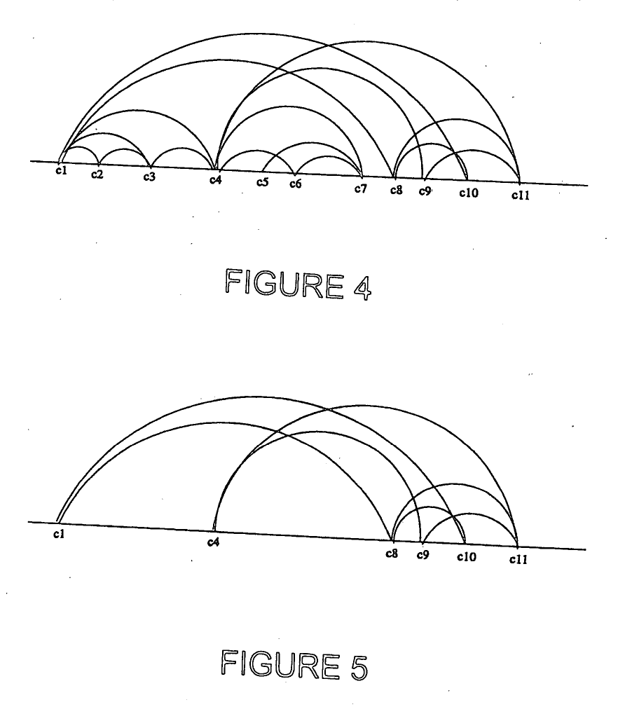

If there are two cores cᵢ, cₖ (i<k) such that ∀ j (i<j<k), N (cⱼ) ε {cᵢ, cᵢ₊₁, . . . , cₖ} and the subgraph formed by cᵢ₊₁, cᵢ₊₂, . . . , cₖ₋₁ is a graph with low topological complexity such as a nested graph, then we can first align segment (cᵢ, cₖ) to the sequence by some algorithm with low computational complexity such as divide-and-conquer or dynamic programming and then treat the whole segment (cᵢ, cₖ) as one edge when formulating the integer program. As shown in the original FIG. 4 and the resultant FIG. 5, segment (c₁, c₄) and segment (c₄, c₈) are reduced to two edges respectively cause the subgraphs formed by c₁, c₂, c₃, c₄ and c₄, c₅, c₆, c₇, c₈ are topologically simple. Segment (c₁, c₄) could be aligned to the sequence by simple dynamic programming algorithm. Segment (c₄, c₈) could be aligned to the sequence by divide-and-conquer algorithm within low degree polynomial time. This kind of graph reduction operation will be formulated as follows.

Definition 7.2 Given a template contact graph CMG=(V, E), |V|≧2, a subset RV={cᵢ₁, cᵢ₂, . . . , cᵢₖ} (i₁<i₂, . . . , <iₖ, k≧2) of V is called Reduced Vertex Set if ∀ e=(cₗ, cₚ) ε (l<p), either at least one of cₗ, cₚ is in RV or there exists one j such that iⱼ<l<p<iⱼ₊₁.

Definition 7.3 Given a template contact graph CMG=(V(CMG), E (CMG)), and its Reduced Vertex Set RV, its Reduced Contact Graph RCMG could be constructed as follows:

1. V(RCMG)=RV;

2. ∀ v₁, v₂ ε V (RCMG), if (v₁, v₂) ε E (CMG), then add (v₁, v₂) to E (RCMG);

3. For any segment (cᵢₙ, cᵢᵣ₊₁), let c, c, . . . , cdenote all cores in RV which are adjacent to at least one core within segment (cᵢₙ, cᵢᵣ₊₁), we add one hyperedge (cᵢₙ cᵢᵣ₊₁, c, c, . . . , c) into E (RCMG).

According to Definition 7.2 and Definition 7.3, we can see that for any given template contact graph, there are often many possible reduced contact graphs (see FIG. 5). To determine which one is the best in terms of computational complexity, let RV={cᵢ₁, cᵢ₂, . . . , cᵢₚ} denote the reduced vertex set of the contact graph after graph reduction operation. Let T_{seg} denote the computational complexity of all segments (cᵢₙ cᵢᵣ₊₁), (r 32 1, 2, . . . , p, when r=p, let r+1=1) and T_{reduced} denote the computational complexity of the integer programming approach on the reduced contact graph. Ideally, the best graph reduction operation should minimize the maximum of T_{seg} and T_{reduced}.

It is easy to theoretically analyse how much time it will take for aligning all segments (see the next subsection). However, it is hard to estimate the computational complexity of the integer programming algorithm on the reduced graph though there is a trivial prediction that T_{reduced}=O (n^{p}) (n is the sequence size.). In practice, T_{reduced} is far smaller than O (n^{p}). There are two factors which are related to the computational time of the integer program. One is the size of the reduced vertex set, and the other is the complexity of the hyperedge, i.e., the number of cores contained in one hyperedge. Real protein data shows that a good reduction is to make T_{seg}=O (M n^{k}), k=3, 4, 5 (M is the number of cores) and limit the complexity of the hyperedge to at most 3. For k=3 or the hyperedge complexity less than 3, the reduced contact graph is still a simple contact graph.

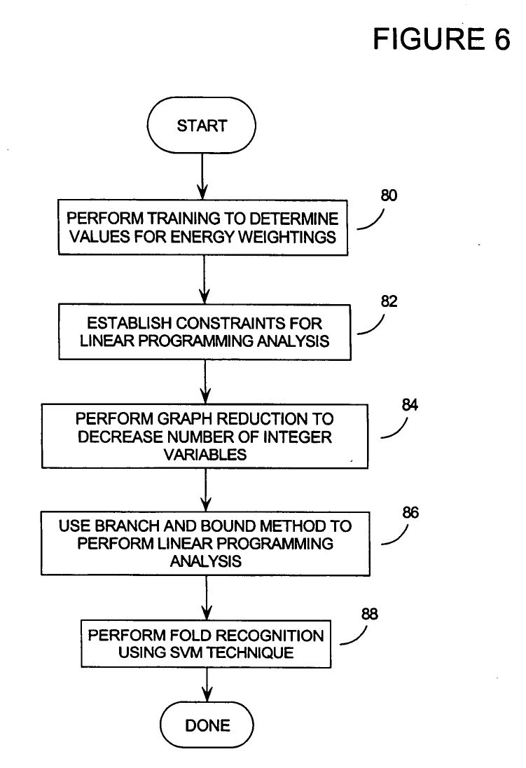

To summarize, the method of the invention can be implemented as presented in the flow chart of FIG. 6.

To begin with, the RAPTOR program uses the energy function described in equations (1)-(7) above. Training is used per step 80, to establish the values of the various energy weightings.

The formulation of linear programming constraints are then determined at step 82. This can be done in a number of ways, including the following:

1. Constraints (8)-(15) could be used;

2. the alternative and improved Constraints (16)-(19), could be used, replacing Constraints (9)-(13) in above item 1; or

3. the further improved formulation, Constraints (20) and (21), could be used, replacing Constraints (9)-(13) in above item 1.

Graph reduction is then performed per step 84, decreasing the number of integer variables and speeding up the LP analysis. The branch and bound method is then used at step 86 to perform the LP analysis. The use of the branch and bound method converts the LP non-integral solutions to integral.

Finally, fold recognition is performed at step 88, using SVM as described above.

While particular embodiments of the present invention have been shown and described, it is clear that changes and modifications may be made to such embodiments without departing from the true scope and spirit of the invention.

The method steps of the invention may be embodied in sets of executable machine codes stored in a variety of formats such as object code or source code. Such code is described generically herein as programming code, or a computer program for simplicity. Clearly, the executable machine code may be integrated with the code of other programs, implemented as subroutines, by external program calls, implemented in the hardware circuit, or by other techniques as known in the art.

The embodiments of the invention may be executed by a computer processor or similar device programmed in the manner of method steps, or may be executed by an electronic system which is provided with means for executing these steps. Similarly, an electronic memory medium such as computer diskettes, CD-Roms, Random Access Memory (RAM), Read Only Memory (ROM) or similar computer software storage media known in the art, may store software code executable to perform such method steps. As well, electronic signals representing these method steps may also be transmitted via a communication network.

The invention could for example be applied to personal computers, super computers, main frames, application service providers (ASPs), Internet servers, smart terminals or personal digital assistants. Again, such implementations would be clear to one skilled in the art, and do not take away from the invention.

What is claimed is:

1. A method of aligning a query protein sequence with a template consisting of a set of pre-selected protein structures in a database, comprising the steps of:
selecting an energy function, said energy function being a sum of energy parameters and weighting factors;
determining values for weighting factors in said energy function;
establishing linear programming (LP) constraints for threading (or aligning) said query protein sequence with each structure in said set of pre-selected protein structures in a database; and
performing a linear programming analysis based on a linear programming formulation including said energy function under said constraints, to optimally align said query protein with said template.

2. The method of claim 1, wherein said step of determining weighting factors comprises the step of using training to determine values for said weighting factors.

3. The method of claim 2, where said energy function comprises the function: min WₘEₘ+WₛEₛ+WₚEₚ+W_{g}E_{g}+WₛₛEₛₛ.

4. The method of claim 1, where alignment gaps are confined to loops.

5. The method of claim 1, where only interaction between core residues is considered.

6. The method of claim 1 wherein said step of performing a linear programming analysis is done on the assumption that solutions are likely to be integral.

7. The method of claim 6, wherein said step of performing a linear programming analysis comprises the step of using a branch and bound technique to perform said linear programming analysis.

8. The method of claim 1, where said linear programming constraints comprise Constraints (8)-(15).

9. The method of claim 1, where said linear programming constraints comprise Constraints (8), (14), (15) and (16)-(19).

10. The method of claim 1, where said linear programming constraints comprise Constraints (8), (14), (15), (20) and (21).

11. The method of claim 1 further comprising the step of performing graph reduction to decrease the number of integer variables and speed up the LP analysis.

12. The method of claim 1, further comprising the step of performing fold analysis using a support vector machine (SVM) algorithm.

13. The method of claim 1, comprising step of generating a dense contact graph prior to said step of performing a linear programming analysis.

14. A method of alignment comprising the steps of:
formulating the protein threading problem as a large scale integer programming problem;
relaxing this problem to a linear programming problem; and solving the integer program by a branch-and-bound method.

15. A system for aligning proteins comprising:
a computer operable to align a query protein sequence with a template consisting of a set of pre-selected protein structures in a database, by performing the steps of:
selecting an energy function;
determining values for weighting factors in said energy function;
establishing linear programming (LP) constraints for threading (or aligning) said query protein sequence with each structure in said set of pre-selected protein structures in a database; and
performing a linear programming analysis based on a linear programming formulation including said energy function under said constraints, to optimally align said query protein with said template.
